(19) United States
(12) Patent Application Publication
(10) Pub. No.: US 2026/0248634 A1
(43) Pub. Date: Aug. 27, 2026

(54) OSTOMY SKIN BARRIER DEVICE

(71) Applicant: ConvaTec Limited, Flintshire (GB)

(72) Inventors: Daniel Hansen, Bikerod (DK); Kristoffer Hansen, Bikerod (DK); Nicole Ricapito, Flintshire (GB); Ethan Chen, Somerville, MA (US); Tanguy Floch, Flintshire (GB)

(21) Appl. No.: 19/650,605

(22) Filed: Apr. 17, 2026

Related U.S. Application Data
(63) Continuation of application No. PCT/GB2024/052656, filed on Oct. 16, 2024.
(60) Provisional application No. 63/590,837, filed on Oct. 17, 2023.

(30) Foreign Application Priority Data
Dec. 7, 2023 (GB) 2318729.7

(51) Int. Cl. A61F 5/443; A61L 24/04

(52) U.S. Cl. CPC A61F 5/443; A61L 24/043; A61L 2400/14

(57) ABSTRACT

The invention provides an ostomy skin barrier device comprising; a front face and a back face wherein at least a portion of the front face comprises a medical adhesive wherein the medical adhesive comprises at least one pre-gelatinized starch or pre-gelatinized modified starch; and an aperture in the skin barrier device capable of at least partially receiving a stoma, in use.

### TECHNICAL FIELD OF THE INVENTION

The present invention relates to an ostomy skin barrier device comprising a medical adhesive, and a medical adhesive.

### BACKGROUND TO THE INVENTION

Medical adhesives typically comprise gelatin, in particular animal derived gelatin, which can have negative effects such as increased risk of viral infection. Medical adhesives comprising gelatin may also have regulated use restrictions for example they cannot be used on breached skin unless they meet the regulations for Class II or Class III devices. Patients who require medical adhesives for long term use, for example patients with stomas, may prefer to use animal free alternatives whilst not compromising the performance of the medical adhesive.

It would therefore be advantageous to provide a gelatin free medical adhesive for long term use, for example for use with ostomy bags. It would be advantageous to provide an ostomy skin barrier device comprising a gelatin free medical adhesive for long term use.

It would be advantageous to provide a skin barrier device comprising a medical adhesive wherein the skin barrier device has good wet integrity, does not erode on exposure to water, and is enzyme resistant so it does not erode on exposure to enzymes. It would be advantageous to provide a skin barrier device for use with an ostomy bag, comprising a medical adhesive wherein the skin barrier device has good wet integrity, does not erode on exposure to water and is enzyme resistant such that the patient's skin is protected from irritation. It would be advantageous to provide a skin barrier device for use with an ileostomy bag, comprising a medical adhesive wherein the skin barrier device has good wet integrity, does not erode on exposure to water and is enzyme resistant such that the patient's skin is protected from enzyme-containing effluent.

It is an aim of embodiments of the invention to overcome one or more problems of the prior art, whether expressly disclosed herein or not.

### SUMMARY OF THE INVENTION

According to a first aspect of the invention there is provided an ostomy skin barrier device comprising; a front face and a back face wherein at least a portion of the front face comprises a medical adhesive wherein the medical adhesive comprises at least one pre-gelatinized starch or pre-gelatinized modified starch; and an aperture in the skin barrier device capable of at least partially receiving a stoma, in use.

Pre-gelatinization is defined as a pre-cooking step wherein the starch or modified starch is heated with water so that the intermolecular bonds within the starch molecules are broken down, and the starch or modified starch is then dried. Pre-gelatinized starch or pre-gelatinized modified starch may be advantageous because pre-gelatinization may improve the interaction of the starch or modified starch with other materials. Pre-gelatinized starch or pre-gelatinized modified starch may be advantageous because it may provide improved wet integrity due to a reduced amount of modified starch washing out of the adhesives in the presence of water. Pre-gelatinized starch or pre-gelatinized modified starch may be advantageous because it may result in the medical adhesive comprising the pre-gelatinized starch or pre-gelatinized modified starch absorbing water at body temperature and thereby preserving the integrity of the medical adhesive on exposure to water.

The pre-gelatinized starch or pre-gelatinized modified starch may comprise no more than 5 wt. % amylose. The pre-gelatinized starch or pre-gelatinized modified starch may comprise no more than 4 wt. %, 3 wt. %, 2 wt. %, 1 wt. %, 0.8 wt. %, 0.6 wt. %, 0.5 wt. %, 0.4 wt. %, 0.3 wt. %, 0.2 wt. % or no more than 0.1 wt. % amylose. The pre-gelatinized starch or pre-gelatinized modified starch may comprise no amylose or substantially no amylose. Pre-gelatinized starch or pre-gelatinized modified starch comprising no amylose or substantially no amylose may comprise branched glucose polymer units (amylopectin) only, and not straight chain glucose polymer units (amylose). Pre-gelatinized starch or pre-gelatinized modified starch comprising a low amount of or no amylose may be advantageous because it may result in an adhesive providing improved wet integrity performance. Pre-gelatinized starch or pre-gelatinized modified starch comprising a low amount of amylose may provide advantageous wet integrity performance due to the linear structure and lower molecular weight of amylose resulting in higher water solubility and therefore reduced wet integrity.

The pre-gelatinized starch or pre-gelatinized modified starch may comprise at least 95 wt. % amylopectin. The pre-gelatinized starch or pre-gelatinized modified starch may comprise at least 96 wt. %, 97 wt. %, 98 wt. % or at least 99 wt. % amylopectin. The pre-gelatinized starch or pre-gelatinized modified starch may comprise 100 wt. % amylopectin. Pre-gelatinized starch or pre-gelatinized modified starch comprising a high amount of amylopectin may be advantageous because it may result in an adhesive providing improved wet integrity performance. Pre-gelatinized starch or pre-gelatinized modified starch comprising a high amount of amylopectin may provide advantageous wet integrity performance due to the branched structure and higher molecular weight of amylopectin resulting in improved stability on exposure to water and therefore improved wet integrity of the medical adhesive.

In some embodiments the medical adhesive comprises less than 5 wt. % gelatin. The medical adhesive may comprise less than 4 wt. %, 3 wt. %, 2 wt. % or less than 1 wt. % gelatin. In preferred embodiments the medical adhesive is substantially free of gelatin. This embodiment may be advantageous because the medical adhesive may have high water absorption properties and good wet integrity such that water or moisture is absorbed from the skin and the medical adhesive does lose its physical form or detach from the skin in use such that the ostomy skin barrier device remains securely attached to a patient in use. This embodiment may be advantageous because it may provide enzyme resistance from stoma effluent. Gelatin free adhesives may be advantageous because they may be worn on breached skin and may assist in ostomy devices, to which the medical adhesive is applied, meeting regulatory requirements in some countries. Gelatin free adhesives may be advantageous because they are animal free and therefore eliminate the risk of viral infections which may originate from the gelatin.

The medical adhesive may comprise at least one pre-gelatinized modified starch. The medical adhesive may comprise at least one pre-gelatinized modified starch wherein the pre-gelatinized modified starch comprises no more than 5 wt. % amylose.

The medical adhesive may comprise at least one pre-gelatinized starch. The medical adhesive may comprise at least one pre-gelatinized starch or pre-gelatinized modified starch wherein the pre-gelatinized starch or pre-gelatinized modified starch comprises no more than 20 wt. % amylose. The medical adhesive may comprise at least one pre-gelatinized starch or pre-gelatinized modified starch wherein the pre-gelatinized starch or pre-gelatinized modified starch comprises no more than 15 wt. %, 10 wt. %, or no more than 5 wt. % amylose.

In some embodiments the pre-gelatinized starch or pre-gelatinized modified starch comprises a crystallinity of no more than 5%. In some embodiments the pre-gelatinized starch or pre-gelatinized modified starch comprises a crystallinity of no more than 4%, 3%, 2%, 1% or no more than 0.5%. In some embodiments the pre-gelatinized starch or pre-gelatinized modified starch comprises substantially 0% crystallinity. Reduced crystallinity may be achieved by the pre-gelatinization step of the pre-gelatinized starch or pre-gelatinized modified starch. Pre-gelatinized starch or pre-gelatinized modified starch comprising a low crystallinity or substantially no crystallinity may be advantageous because it may result in the pre-gelatinized starch or pre-gelatinized modified starch absorbing water at body temperature and thereby preserving the integrity of the medical adhesive on exposure to water.

The pre-gelatinized starch or pre-gelatinized modified starch may be cross-linked. The pre-gelatinized starch or pre-gelatinized modified starch may be cross-linked with an organic molecule. The pre-gelatinized starch or pre-gelatinized modified starch may be cross-linked with an organic molecule comprising at least three carbons. The pre-gelatinized starch or pre-gelatinized modified starch may be cross-linked with a dialdehyde or a dicarboxylic acid comprising at least three carbons. The pre-gelatinized starch or pre-gelatinized modified starch may be cross-linked with an adipate. In some embodiments the pre-gelatinized starch or pre-gelatinized modified starch is cross-linked with a phosphorus compound. The pre-gelatinized starch or pre-gelatinized modified starch may be cross-linked with phosphorus oxychloride. In some embodiments the pre-gelatinized starch or pre-gelatinized modified starch is not cross-linked. In some embodiments the pre-gelatinized starch or pre-gelatinized modified starch is not cross-linked with glyoxal.

The pre-gelatinized starch or pre-gelatinized modified starch may originate from potatoes, corn (or maize), wheat or tapioca.

The modified starch or the pre-gelatinized modified starch may be selected from the group consisting of: roasted starch with hydrochloric acid, alkaline-modified starch, bleached starch, oxidized starch, enzyme-treated starch, distarch phosphate, hydroxypropylated starch, starch ether, hydroxyethyl starch, cationic starch and carboxymethylated starch, or any combinations thereof.

In preferred embodiments the pre-gelatinized modified starch is a pre-gelatinized esterified starch. The pre-gelatinized modified starch may be a pre-gelatinized oxidised starch. The pre-gelatinized modified starch may be a pre-gelatinized distarch. The pre-gelatinized modified starch may be a pre-gelatinized distarch phosphate. The pre-gelatinized modified starch may be pre-gelatinized acetylated distarch phosphate. The pre-gelatinized modified starch may be pre-gelatinized hydroxypropylated distarch phosphate. The pre-gelatinized modified starch may be a pre-gelatinized distarch adipate. The pre-gelatinized modified starch may be a pre-gelatinized acetylated distarch adipate. The pre-gelatinized acetylated distarch adipate may be of the formula:

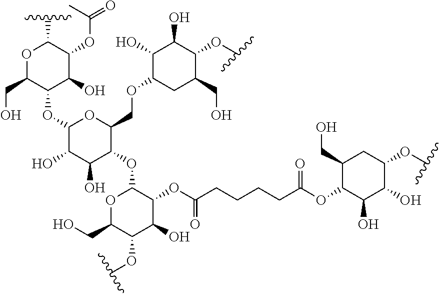

The pre-gelatinized acetylated distarch adipate may be sourced from a waxy maize. The pre-gelatinized acetylated distarch adipate may comprise no more than 5% crystallinity, preferably approximately 0% crystallinity. The pre-gelatinized acetylated distarch adipate may comprise substantially no amylose. The medical adhesive comprising pre-gelatinized acetylated distarch adipate may be advantageous because it may provide improved wet integrity performance and improved enzyme resistance compared to medical adhesives comprising gelatin. The gelatin free medical adhesive comprising pre-gelatinized acetylated distarch adipate may be advantageous because it may provide comparable wet integrity and improved enzyme resistance compared to medical adhesives comprising gelatin.

In alternative embodiments the pre-gelatinized hydroxypropylated distarch phosphate may comprise no more than 5 wt. % or substantially no amylose. The medical adhesive comprising pre-gelatinized hydroxypropylated distarch phosphate may be advantageous because it may provide improved wet integrity performance and improved enzyme resistance compared to medical adhesives comprising gelatin. The gelatin free medical adhesive comprising pre-gelatinized hydroxypropylated distarch phosphate may be advantageous because it may provide comparable wet integrity and improved enzyme resistance compared to medical adhesives comprising gelatin.

In alternative preferred embodiments the pre-gelatinized starch is pre-gelatinized potato starch. The pre-gelatinized potato starch may comprise no more than 25% amylose. The pre-gelatinized potato starch may comprise no more than 20%, 15%, 10% or no more than 5% amylose.

The amount of pre-gelatinized starch or pre-gelatinized modified starch may be between 10 wt. % and 40 wt. % of the total weight of the medical adhesive. The amount of pre-gelatinized starch or pre-gelatinized modified starch may be between 10 wt. % and 35 wt. %, 10 wt. % and 30 wt. %, 15 wt. % and 40 wt. %, 15 wt. % and 35 wt. %, 15 wt. % and 30 wt. %, 20 wt. % and 40 wt. %, 20 wt. % and 35 wt. % or between 20 wt. % and 30 wt. %, of the total weight of the medical adhesive.

The medical adhesive may comprise pectin and/or a pectin derivative. The pectin may be high methoxyl pectin (at least 50% esterified). The pectin may be between 50% and 80% esterified, or between 50% and 70%, or between 55% and 65% esterified. The pectin may be low methoxyl pectin (less than 50% esterified). The pectin derivative may comprise pectin modified by at least one of the methods selected from the group consisting of: alkylation, amidation, quaternization, thiolation, sulfation, oxidation, chain elongation and depolymerization or any combination thereof.

The amount of pectin or pectin derivative may be between 10 wt. % and 30 wt. % of the total weight of the medical adhesive. The amount of pectin or pectin derivative may be between 12 wt. % and 28 wt. %, 12 wt. % and 26 wt. %, 12 wt. % and 25 wt. %, or between 13 wt. % and 25 wt. % of the total weight of the medical adhesive. The amount of pectin or pectin derivative may be between 14 wt. % and 26 wt. %, 15 wt. % and 25 wt. %, 16 wt. % and 25 wt. %, 18 wt. % and 25 wt. %, 20 wt. % and 25 wt. %, 18 wt. % and 23 wt. %, 18 wt. % and 22 wt. % or between 19 wt. % and 21 wt. % of the total weight of the medical adhesive. In some embodiments the amount of pectin or pectin derivative may be between 18 and 20 wt. % of the total weight of the medical adhesive. In alternative embodiments the amount of pectin or pectin derivative may be between 12 and 15 wt. % of the total weight of the medical adhesive.

The medical adhesive may comprise polyisobutylene. The medical adhesive may comprise at least one polyisobutylene. The medical adhesive may comprise two polyisobutylene compounds. The polyisobutylene may have an average molecular weight of between 50,000 gmol⁻¹ and 110,000 gmol⁻¹. The polyisobutylene may have an average molecular weight of between 500,000 gmol⁻¹ and 1,200,000 gmol⁻¹.

The amount of polyisobutylene may be between 5 wt. % and 50 wt. % of the total weight of the medical adhesive. In some embodiments the amount of polyisobutylene is between 5 wt. % and 45 wt. %, 5 wt. % and 40 wt. % or between 5 wt. % and 35 wt. % of the total weight of the medical adhesive. In some embodiments the amount of polyisobutylene is between 30 wt. % and 48 wt. %, 34 wt. % and 46 wt. %, 35 wt. % and 45 wt. %, 36 wt. % and 45 wt. %, 38 wt. % and 45 wt. %, 30 wt. % and 45 wt. %, 38 wt. % and 43 wt. %, 38 wt. % and 42 wt. % or between 39 wt. % and 41 wt. % of the total weight of the medical adhesive. In some embodiments the amount of polyisobutylene is approximately 40 wt. % of the total weight of the medical adhesive. In alternative embodiments the amount of polyisobutylene is between 5 wt. % and 30 wt. %, 5 wt. % and 25 wt. %, 5 wt. % and 20 wt. %, 5 wt. % and 15 wt. % or between 5 wt. % and 10 wt. % of the total weight of the medical adhesive. In some embodiments the amount of polyisobutylene is approximately 8 wt. % of the total weight of the medical adhesive.

The medical adhesive may comprise cellulose or a cellulose derivative. The cellulose derivative may be a modified cellulose. The cellulose derivative may be methylcellulose. The cellulose derivative may be hydroxypropyl cellulose. In preferred embodiments the cellulose derivative is carboxymethyl cellulose.

The amount of cellulose or a cellulose derivative may be between 10 wt. % and 30 wt. % of the total weight of the medical adhesive. The amount of cellulose or a cellulose derivative may be between 12 wt. % and 28 wt. %, 14 wt. % and 26 wt. %, 14 wt. % and 25 wt. %, 14 wt. % and 24 wt. %, 14 wt. % and 22 wt. %, 15 wt. % and 25 wt. %, 16 wt. % and 25 wt. %, 18 wt. % and 25 wt. %, 20 wt. % and 25 wt. %, 18 wt. % and 23 wt. %, 18 wt. % and 22 wt. % or between 19 wt. % and 21 wt. % of the total weight of the medical adhesive. In some embodiments the amount of cellulose or a cellulose derivative is approximately 20 wt. % of the total weight of the medical adhesive. In alternative embodiments the amount of cellulose or a cellulose derivative is approximately 15 wt. % of the total weight of the medical adhesive.

The medical adhesive may comprise at least one additional component selected from the group consisting of: acrylate or acrylic, epoxy, silicone, polyurethane, natural rubber, styrene isoprene styrene copolymer (SIS or SIS/SI), butyl rubber, hydrocolloids, binders, tack enhancers, mineral oil, antioxidants and hydrogels or any combination thereof.

In some embodiments the medical adhesive comprises butyl rubber. Butyl rubber may comprise at least 95 wt. %, 96 wt. %, 97 wt. % or at least 98 wt. % polyisobutylene. The medical adhesive may comprise between 5 wt. % and 30 wt. % butyl rubber. The medical adhesive may comprise between 5 wt. % and 25 wt. %, 5 wt. % and 20 wt. %, or between 10 wt. % and 20 wt. % butyl rubber.

The medical adhesive may comprise between 3 wt. % and 15 wt. % styrene-isoprene-styrene copolymer. The medical adhesive may comprise between 5 wt. % and 15 wt. % styrene-isoprene-styrene copolymer.

The medical adhesive may comprise between 5 wt. % and 20 wt. % tackifier. The medical adhesive may comprise between 10 wt. % and 20 wt. % or between 5 and 15 wt. % tackifier.

The medical adhesive may comprise between 5 wt. % and 20 wt. % mineral oil. The medical adhesive may comprise between 5 wt. % and 15 wt. % or between 10 and 20 wt. % mineral oil.

The medical adhesive may comprise; between 10 and 40 wt. % pre-gelatinized starch or pre-gelatinized modified starch; between 10 and 30 wt. % pectin or pectin derivative; between 5 and 50 wt. % polyisobutylene; and between 10 and 30 wt. %. cellulose or cellulose derivative.

The medical adhesive may comprise; between 10 and 25 wt. % pre-gelatinized starch or pre-gelatinized modified starch; between 10 and 20 wt. % pectin or pectin derivative; between 5 and 50 wt. % polyisobutylene; and between 10 and 30 wt. %. cellulose or cellulose derivative.

The medical adhesive may comprise; between 10 and 40 wt. % pre-gelatinized starch or pre-gelatinized modified starch; between 10 and 30 wt. % pectin or pectin derivative; between 30 and 50 wt. % polyisobutylene; and between 10 and 30 wt. %. cellulose or cellulose derivative.

The medical adhesive may comprise; between 15 and 35 wt. % pre-gelatinized starch or pre-gelatinized modified starch; between 10 and 30 wt. % pectin or pectin derivative; between 30 and 50 wt. % polyisobutylene; and between 10 and 30 wt. %. cellulose or cellulose derivative.

The medical adhesive may comprise; between 10 and 40 wt. % pre-gelatinized starch or pre-gelatinized modified starch; between 15 and 25 wt. % pectin or pectin derivative; between 30 and 50 wt. % polyisobutylene; and between 10 and 30 wt. %. cellulose or cellulose derivative.

The medical adhesive may comprise; between 10 and 40 wt. % pre-gelatinized starch or pre-gelatinized modified starch; between 10 and 30 wt. % pectin or pectin derivative; between 35 and 45 wt. % polyisobutylene; and between 10 and 30 wt. %. cellulose or cellulose derivative.

The medical adhesive may comprise; between 10 and 40 wt. % pre-gelatinized starch or pre-gelatinized modified starch; between 10 and 30 wt. % pectin or pectin derivative; between 30 and 50 wt. % polyisobutylene; and between 15 and 25 wt. %. cellulose or cellulose derivative.

The medical adhesive may comprise; between 15 and 35 wt. % pre-gelatinized starch or pre-gelatinized modified starch; between 15 and 25 wt. % pectin or pectin derivative; between 35 and 45 wt. % polyisobutylene; and between 15 and 25 wt. %. cellulose or cellulose derivative.

The medical adhesive may comprise; approximately 20 wt. % pre-gelatinized starch or pre-gelatinized modified starch; approximately 20 wt. % pectin or pectin derivative; approximately 40 wt. % polyisobutylene; and approximately 20 wt. %. cellulose or cellulose derivative.

The medical adhesive may comprise; approximately 35 wt. % pre-gelatinized starch or pre-gelatinized modified starch; approximately 20 wt. % pectin or pectin derivative; approximately 40 wt. % polyisobutylene; and approximately 20 wt. %. cellulose or cellulose derivative.

The medical adhesive may comprise; between 10 and 20 wt. % pre-gelatinized starch or pre-gelatinized modified starch; between 10 and 20 wt. % pectin or pectin derivative; between 5 and 20 wt. % polyisobutylene; and between 10 and 20 wt. %. cellulose or cellulose derivative.

In some embodiments at least 50% of the front face of the ostomy skin barrier device comprises the medical adhesive. In some embodiments at least 60%, 70%, 80%, 85%, 90%, 95%, 96%, 97%, 98% or at least 99% of the front face of the ostomy skin barrier device comprises the medical adhesive. In some embodiments between 50% and 99% of the front face of the ostomy skin barrier device comprises the medical adhesive. In some embodiments between 55% and 99%, 65% and 99%, 70% and 99%, 70% and 95%, 70% and 90% or between 80% and 90% of the front face of the ostomy skin barrier device comprises the medical adhesive.

The medical adhesive may be in the form of a layer. The thickness of the adhesive layer may be between 0.05 mm and 2 mm. The thickness of the adhesive layer may be between 0.4 mm and 2 mm. The thickness of the adhesive layer may be between 0.5 mm and 2 mm or between 0.6 and 2 mm. In some embodiments the thickness of the adhesive layer may be between 0.05 mm and 0.4 mm. The thickness of the adhesive layer may be between 0.1 mm and 0.4 mm, 0.05 mm and 0.35 mm or between 0.05 and 0.3 mm.

In some embodiments the ostomy skin barrier device comprises a solution-cast hydrocolloid tape collar. The ostomy skin barrier device may comprise a hydrocolloid tape collar wherein the thickness of the adhesive layer comprising the medical adhesive may be between 0.01 mm and 0.4 mm, 0.05 mm and 0.35 mm or between 0.05 and 0.3 mm. The ostomy skin barrier device may comprise a solution-cast hydrocolloid tape collar wherein the thickness of the adhesive layer comprising the medical adhesive may be between 0.01 mm and 0.4 mm, 0.05 mm and 0.35 mm or between 0.05 and 0.3 mm.

In some embodiments the medical adhesive is a gel. In alternative embodiments the medical adhesive is a powder. In some embodiments the medical adhesive is a powder and the medical adhesive may comprise no more than 1 wt. % polyisobutylene. In some embodiments the medical adhesive is a powder and the medical adhesive may comprise no more than 0.5 wt. %, 0.4 wt. %, 0.3 wt. %, 0.2 wt. % or no more than 0.1 wt. % polyisobutylene.

The front face of the ostomy skin barrier device may comprise a second medical adhesive. The second medical adhesive may be an acrylic adhesive. The second medical adhesive may be located towards at least one edge of the ostomy skin barrier device. The second medical adhesive may be located towards at least one outer edge of the ostomy skin barrier device. The second medical adhesive may cover no more than 40% of the front face of the ostomy skin barrier device. The second medical adhesive may cover no more than 35%, 30%, 25%, 20%, 15% or no more than 10% of the front face of the ostomy skin barrier device.

The ostomy skin barrier device may comprise a polymer or polymeric matrix. The ostomy skin barrier device may comprise an elastomer. The ostomy skin barrier device may comprise a thermoelastic polymer.

The aperture may be mouldable to the size of the stoma. The ostomy skin barrier device may be mouldable to the size of the stoma. The ostomy skin barrier device may be mouldable to cover uneven skin surrounding or located next to the stoma.

The ostomy skin barrier device may be connected to an ostomy bag. The ostomy skin barrier device may be detachably connected to an ostomy bag.

In alternative embodiments the ostomy skin barrier device may not be connected to an ostomy bag and instead, in use, may be located between the ostomy bag and the patient's skin to protect the peristomal skin. In alternative embodiments the ostomy skin barrier device may not be connected to an ostomy bag and instead, in use, may be located between a skin barrier wafer and the patient's skin to protect the peristomal skin.

The ostomy skin barrier device may comprise a plate or sheet arranged in use to connect to an ostomy bag or pouch. The ostomy skin barrier device may comprise a connector, arranged in use to connect to an ostomy bag or pouch.

In preferred embodiments there is provided an ostomy skin barrier device comprising; a front face and a back face wherein at least a portion of the front face comprises a medical adhesive wherein the medical adhesive comprises a pre-gelatinized starch or a pre-gelatinized modified starch; and an aperture in the skin barrier device for at least partially receiving a stoma, in use, wherein the pre-gelatinized starch or the pre-gelatinized modified starch comprises no more than 5 wt. % amylose.

In preferred embodiments there is provided an ostomy skin barrier device comprising; a front face and a back face wherein at least a portion of the front face comprises a medical adhesive wherein the medical adhesive comprises a pre-gelatinized starch or a pre-gelatinized modified starch; and an aperture in the skin barrier device for at least partially receiving a stoma, in use, wherein the pre-gelatinized starch or the pre-gelatinized modified starch comprises at least 95 wt. % amylopectin.

In preferred embodiments there is provided an ostomy skin barrier device comprising; a front face and a back face wherein at least a portion of the front face comprises a medical adhesive wherein the medical adhesive comprises a pre-gelatinized starch or a pre-gelatinized modified starch; and an aperture in the skin barrier device for at least partially receiving a stoma, in use, wherein the pre-gelatinized starch or pre-gelatinized modified starch is a pre-gelatinized distarch.

In preferred embodiments there is provided an ostomy skin barrier device comprising; a front face and a back face wherein at least a portion of the front face comprises a medical adhesive wherein the medical adhesive comprises a pre-gelatinized starch or a pre-gelatinized modified starch; and an aperture in the skin barrier device for at least partially receiving a stoma, in use, wherein the pre-gelatinized starch or pre-gelatinized modified starch is a pre-gelatinized distarch adipate.

In preferred embodiments there is provided an ostomy skin barrier device comprising; a front face and a back face wherein at least a portion of the front face comprises a medical adhesive wherein the medical adhesive comprises a pre-gelatinized starch or a pre-gelatinized modified starch; and an aperture in the skin barrier device for at least partially receiving a stoma, in use, wherein the pre-gelatinized starch or pre-gelatinized modified starch is a pre-gelatinized distarch adipate or a pre-gelatinized distarch phosphate.

In preferred embodiments there is provided an ostomy skin barrier device comprising; a front face and a back face wherein at least a portion of the front face comprises a medical adhesive wherein the medical adhesive comprises a pre-gelatinized starch or a pre-gelatinized modified starch; and an aperture in the skin barrier device for at least partially receiving a stoma, in use, wherein the pre-gelatinized starch or pre-gelatinized modified starch is a pre-gelatinized acetylated distarch adipate or a pre-gelatinized hydroxypropyl distarch phosphate.

According to a second aspect of the invention there is provided a medical adhesive comprising at least one starch or modified starch wherein the starch or modified starch comprises no more than 5 wt. % amylose.

The starch or modified starch may comprise no more than 4 wt. %, 3 wt. %, 2 wt. %, 1 wt. %, 0.8 wt. %, 0.6 wt. %, 0.5 wt. %, 0.4 wt. %, 0.3 wt. %, 0.2 wt. % or no more than 0.1 wt. % amylose.

Any of the embodiments of the medical adhesive of the ostomy skin barrier device of the first aspect of the invention may apply to the second aspect of the invention with the exception that the starch or modified starch of the second aspect of the invention may be pre-gelatinized or may not be pre-gelatinized.

In preferred embodiments there is provided a medical adhesive comprising at least one starch or modified starch wherein the starch or modified starch comprises no more than 5 wt. % amylose, and wherein the starch or modified starch is a pre-gelatinized distarch.

In preferred embodiments there is provided a medical adhesive comprising at least one starch or modified starch wherein the starch or modified starch comprises no more than 5 wt. % amylose, and wherein the starch or modified starch is a pre-gelatinized distarch adipate or pre-gelatinized distarch phosphate.

In preferred embodiments there is provided a medical adhesive comprising at least one starch or modified starch wherein the starch or modified starch comprises no more than 5 wt. % amylose, and wherein the starch or modified starch is a pre-gelatinized acetylated distarch adipate or a pre-gelatinized hydroxypropyl distarch phosphate.

According to a third aspect of the invention there is provided a medical adhesive comprising at least one starch or modified starch wherein the starch or modified starch comprises a pre-gelatinized potato starch.

Any of the embodiments of the medical adhesive of the ostomy skin barrier device of the first aspect of the invention may apply to the third aspect of the invention with the exception that the starch or modified starch of the third aspect of the invention comprises a pre-gelatinized potato starch.

According to a fourth aspect of the invention there is provided a medical adhesive comprising at least one starch or modified starch wherein the starch or modified starch comprises at least 95 wt. % amylopectin.

The one starch or modified starch wherein the starch or modified starch may comprise at least 96%, 97%, 98% or at least 99% amylopectin. The starch or modified starch may comprise 100% amylopectin.

Any of the embodiments of the medical adhesive of the ostomy skin barrier device of the first aspect of the invention may apply to the fourth aspect of the invention with the exception that the starch or modified starch of the fourth aspect of the invention may be pre-gelatinized or may not be pre-gelatinized.

In preferred embodiments there is provided a medical adhesive comprising at least one starch or modified starch wherein the starch or modified starch comprises at least 95 wt. % amylopectin, and wherein the starch or modified starch is a pre-gelatinized distarch.

In preferred embodiments there is provided a medical adhesive comprising at least one starch or modified starch wherein the starch or modified starch comprises at least 95 wt. % amylopectin, and wherein the starch or modified starch is a pre-gelatinized distarch adipate or pre-gelatinized distarch phosphate.

In preferred embodiments there is provided a medical adhesive comprising at least one starch or modified starch wherein the starch or modified starch comprises at least 95 wt. % amylopectin, and wherein the starch or modified starch is a pre-gelatinized acetylated distarch adipate or a pre-gelatinized hydroxypropyl distarch phosphate.

The medical adhesive of the second, third or fourth aspects of the invention may be in contact with a backing material. The backing material may comprise a material selected from the group consisting of: a fibrous material, a hydrogel, modified cellulose, alginate, carboxymethyl cellulose, a foam and a polymer such as polyurethane (PU), a polyvinyl chloride (PVC) a silicone elastomer or a fluoropolymer, and any combination thereof. This embodiment may be advantageous because it may allow the adhesive to be used in a wound dressing wherein the wound dressing has improved wet integrity, water absorption, peel and tack performance in use.

The medical adhesive of the second, third or fourth aspects of the invention may be a gel. The medical adhesive of the second, third or fourth aspects of the invention may be a solid. The medical adhesive of the second, third or fourth aspects of the invention may be a powder.

In some embodiments the medical adhesive of the second, third or fourth aspects of the invention may be a powder and may comprise no more than 1 wt. % polyisobutylene. The medical adhesive of the second, third or fourth aspects of the invention may be a powder and may comprise no more than 0.5 wt. %, 0.4 wt. %, 0.3 wt. %, 0.2 wt. % or no more than 0.1 wt. % polyisobutylene.

According to a fifth aspect of the invention there is provided an ostomy skin barrier device comprising; a front face and a back face wherein at least a portion of the front face comprises a medical adhesive wherein the medical adhesive is according to the second, third or fourth aspect of the invention; and an aperture in the skin barrier device capable of at least partially receiving a stoma, in use.

Any of the embodiments of the ostomy skin barrier device of the first aspect of the invention may apply to the fifth aspect of the invention with the exception that the adhesive is according to the second, third or fourth aspect of the invention.

According to a sixth aspect of the invention there is provided an ostomy bag comprising an ostomy skin barrier device of the first or the fifth aspect of the invention. The ostomy skin barrier device may be connected to the ostomy bag or may be integrally formed with the ostomy bag.

According to a seventh aspect of the invention there is provided a kit comprising an ostomy skin barrier device of the first or the fifth aspect of the invention and an ostomy bag. The ostomy bag and ostomy skin barrier device may be packaged in a suitable container, such as a bag or pouch, for example.

### DETAILED DESCRIPTION OF THE INVENTION

In order that the invention may be more clearly understood embodiments thereof will now be described, by way of example only, with reference to the accompanying drawings, of which:

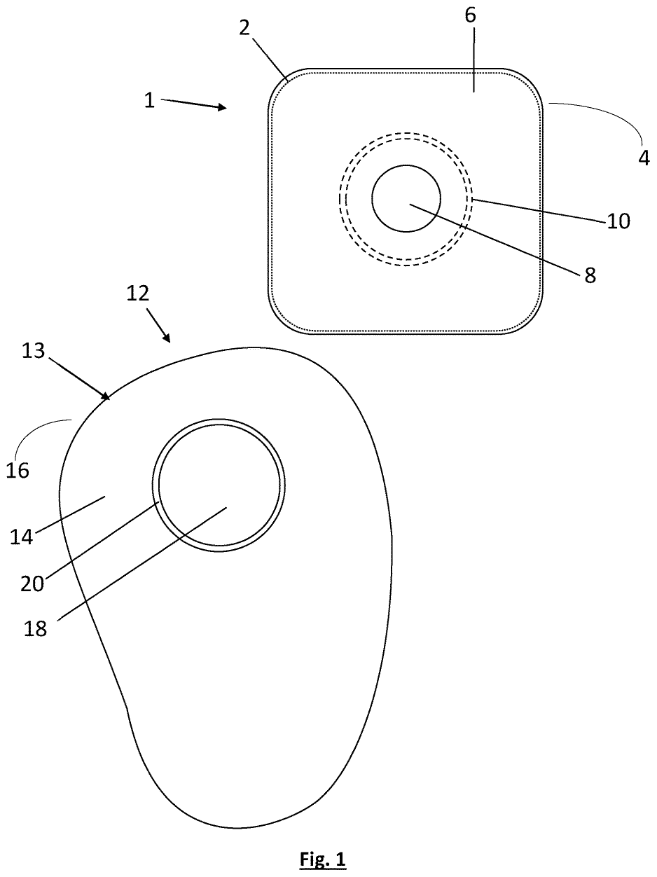

FIG. 1 illustrates a first embodiment of an ostomy skin barrier device (1) of the first aspect of the invention and an ostomy bag (12).

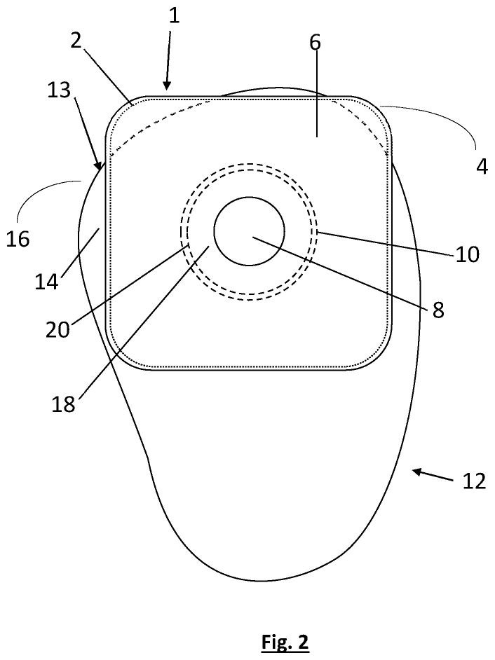

FIG. 2 illustrates the first embodiment of an ostomy skin barrier device (1) of the first aspect of the invention and the ostomy bag (12) of FIG. 1 whereby the ostomy skin barrier device (1) and the ostomy bag (12) are connected together.

FIG. 3 shows a graph indicating the amount of water (Δm) that a medical adhesive (6) of the first embodiment of the ostomy bag of the first aspect comprising inventive adhesive A, inventive adhesive B or control gelatin adhesive X absorbed over 360 minutes at 32° C.

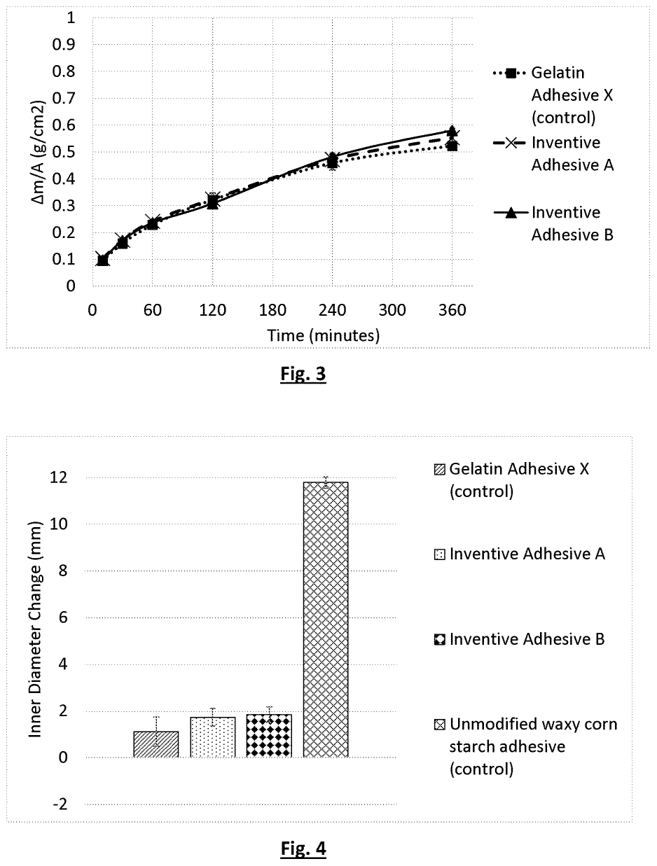

FIG. 4 shows a graph indicating the inner diameter change of the medical adhesive (6) of the first embodiment of the ostomy skin barrier device (1) of the first aspect of the invention, where the medical adhesive (6) comprises inventive adhesive A, inventive adhesive B, control gelatin adhesive X, or control unmodified waxy corn starch that is not pre-gelatinized, after exposure to saline.

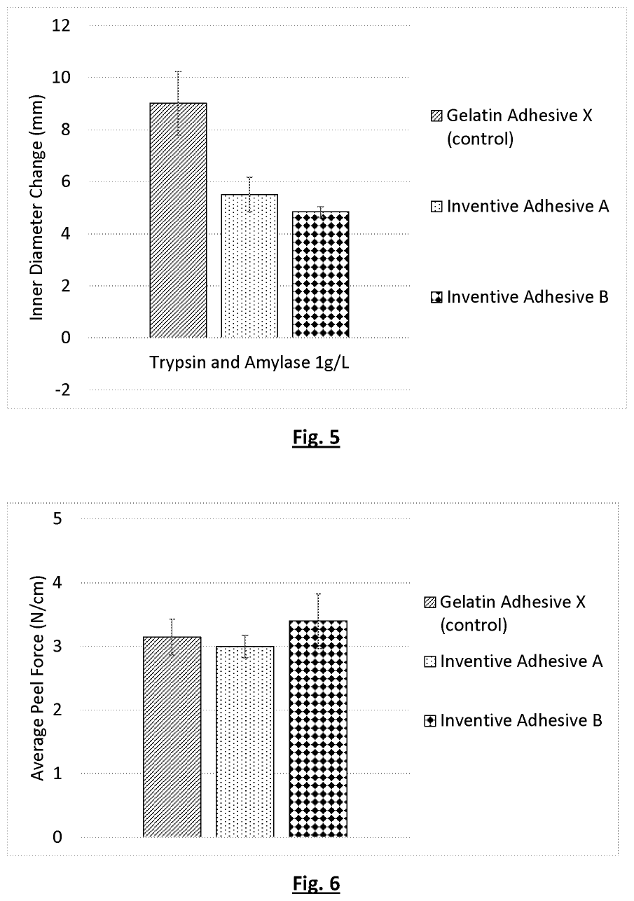

FIG. 5 shows a graph indicating the inner diameter change of the medical adhesive (6) of the first embodiment of the ostomy skin barrier device (1) of the first aspect of the invention where the medical adhesive (6) comprises inventive adhesive A, inventive adhesive B, or control gelatin adhesive X after exposure to a mixture comprising 1 g/L amylase and 1 g/L trypsin in saline.

FIG. 6 shows a graph indicating the average peel force of inventive adhesive A, inventive adhesive B or control gelatin adhesive X.

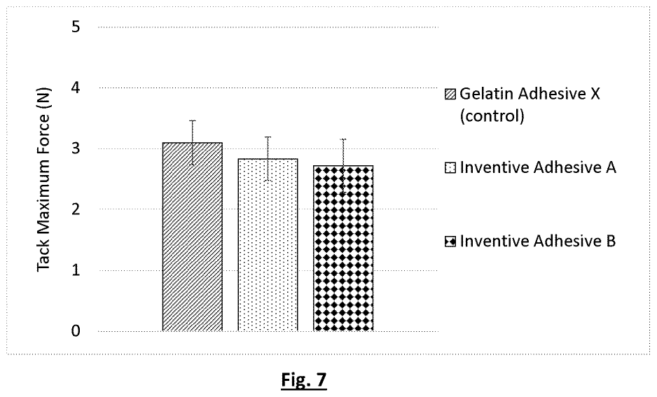

FIG. 7 shows a graph indicating the maximum tack force of inventive adhesive A, inventive adhesive B or control gelatin adhesive X.

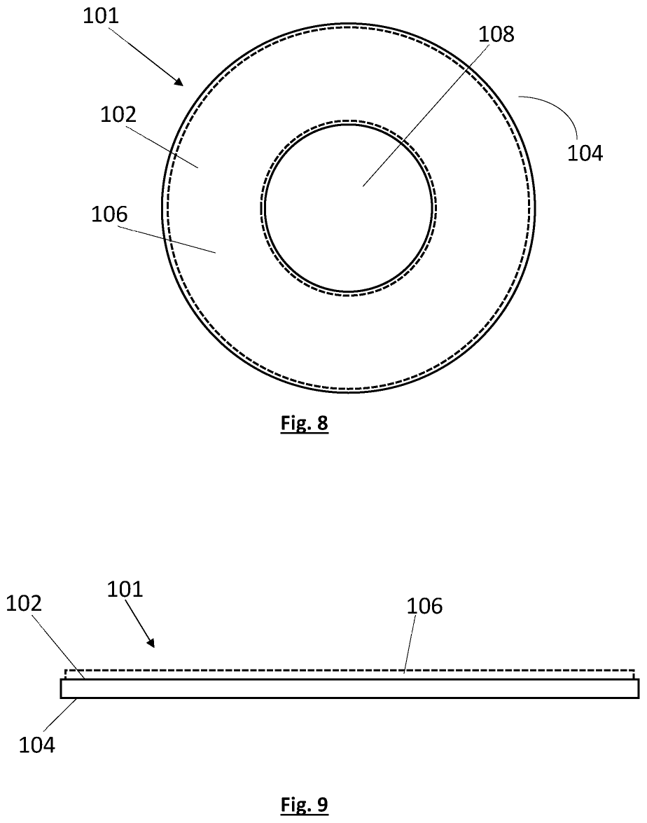

FIG. 8 illustrates a second embodiment of an ostomy skin barrier device (101) of the first aspect of the invention.

FIG. 9 illustrates a cross-sectional view of the second embodiment of an ostomy skin barrier device (101) of the first aspect of the invention of FIG. 8.

A first embodiment of an ostomy skin barrier device (1) of the first aspect of the invention and an ostomy bag (12) are illustrated in FIG. 1. The skin barrier device (1) comprises; a front face (2) and an opposing back face (4) wherein at least a portion or substantially all of the front face (2) comprises a medical adhesive (6), illustrated by a dotted line, wherein the medical adhesive (6) comprises a pre-gelatinized modified starch. The ostomy skin barrier device (1) comprises an aperture (8) suitable for at least partially receiving a stoma.

The back face (4) comprises a skin barrier device connector (10) surrounding the aperture (8) wherein the skin barrier device connector (10) is suitable for connecting to the corresponding ostomy bag connector (20) located on the ostomy bag (12).

The ostomy bag (12) comprises a pouch (13) comprising a front panel (14) and a back panel (16). The front panel (14) comprises an aperture (18) capable of at least partially receiving a stoma, in use. The aperture (18) is surrounded by the ostomy bag connector (20) which is suitable for connecting to the corresponding skin barrier device connector (10).

In use, the ostomy skin barrier device (1) is connected to the ostomy bag (12) as illustrated in FIG. 2. The front face (2) of the ostomy skin barrier device (1) is in contact with and adhered to the patient's peristomal skin by way of the medical adhesive (6). The back face (4) of the ostomy skin barrier device (1) is therefore facing away from the patient's skin. The skin barrier device connector (10) is connected to the corresponding ostomy bag connector (20) such that the back face (4) of the ostomy skin barrier device (1) is in contact with the front panel (14) of the ostomy bag (12). The patient's stoma passes through the ostomy skin barrier device aperture (8) and the ostomy bag aperture (18) such that effluent can pass into the pouch (13). The back panel (16) of the ostomy bag (12) is facing away from the patient's body.

### Example Medical Adhesives

Example medical adhesives (6) were prepared according to formulations in table 1 wherein the modified pre-gelatinized starch is pre-gelatinized acetylated distarch adipate (inventive adhesive A) or pre-gelatinized hydroxypropyl distarch phosphate (inventive adhesive B). Inventive adhesive A and inventive adhesive B are substantially gelatin free.

Control adhesives comprising gelatin (gelatin adhesive X) or unmodified waxy corn starch were also prepared for comparison.

TABLE 1

| | | | | Unmodified |
|---|---|---|---|---|
| | | | Gelatin | waxy corn |
| | Inventive | Inventive | adhesive | starch |
| | adhesive | adhesive | X | adhesive |
| | A | B | (control) | (control) |
| pre-gelatinized | 20 | | | |
| acetylated | | | | |
| distarch | | | | |
| adipate, wt. % | | | | |
| pre-gelatinized | | 20 | | |
| hydroxypropyl | | | | |
| distarch | | | | |
| phosphate, | | | | |
| wt. % | | | | |
| Gelatin, wt. % | | | 20 | |
| Unmodified | | | | 20 |
| waxy corn | | | | |
| starch, wt. % | | | | |
| Pectin, wt. % | 20 | 20 | 20 | 20 |
| Polyiso- | 40 | 40 | 40 | 40 |
| butylene, wt. % | | | | |
| Carboxymethyl | 20 | 20 | 20 | 20 |
| cellulose, wt. % | | | | |

The pre-gelatinized acetylated distarch adipate comprises at least 95 wt. % amylopectin and no more than 5 wt. % amylose.

The pre-gelatinized hydroxypropyl distarch phosphate comprises at least 95 wt. % amylopectin and no more than 5 wt. % amylose.

The unmodified waxy corn starch is not pre-gelatinized. The unmodified waxy corn starch comprises at least 95 wt. % amylopectin and no more than 5 wt. % amylose. The unmodified waxy corn starch used is Sigma-Aldrich® product S9679.

The water absorption, the wet integrity and the enzyme resistance of the medical adhesive (6) comprising the inventive formulations of table 1 were tested and compared to the medical adhesive (6) comprising control gelatin adhesive X. The wet integrity of the medical adhesive (6) was also compared to control adhesive comprising unmodified waxy corn starch that is not pre-gelatinized.

The wet integrity test tests the ability of the medical adhesive (6) to maintain its physical form when exposed to fluid, in this case saline. The wet integrity test was carried out according to the ISO 12505-2:2016 method. The enzyme resistance test tests the ability of the medical adhesive (6) to maintain its physical form when exposed to enzymes. The enzyme resistance test was carried according to a modified ISO 12505-2:2016 method wherein the enzymes amylose and trypsin were added to saline at a concentration of 1 g/L each.

FIG. 3 shows the amount of water that inventive adhesive A, inventive adhesive B and control gelatin adhesive X absorbed (Δm) within 360 minutes at 32° C. The data shows that the performance of inventive adhesive A and inventive adhesive B is comparable to control gelatin adhesive X. This shows that replacing the gelatin with pre-gelatinized acetylated distarch adipate or to pre-gelatinized hydroxypropyl distarch phosphate in the medical adhesive (6) does not negatively impact the water absorption properties of the medical adhesive (6). In use this results in a gelatin free adhesive which maintains adhesion on exposure to water and therefore results in longer wear times.

FIG. 4 shows the wet integrity performance of the medical adhesive (6) by showing the change in the diameter of the aperture (8) of the ostomy device (1) (inner diameter change) when the medical adhesive (6) comprises inventive adhesive A or inventive adhesive B after exposure to saline at 32° C. The data was compared to control gelatin adhesive X, and control unmodified waxy corn starch adhesive that is not pre-gelatinized. Negative inner diameter change shows that the adhesive swells around the stoma (sealing the stoma). Positive inner diameter change is equivalent to erosion and shows that the adhesive erodes on exposure to saline. The results show that the wet integrity performance of inventive adhesives A and B is comparable to the wet integrity performance of control gelatin adhesive X and superior to the control unmodified waxy corn starch adhesive that is not pre-gelatinized. The control unmodified waxy corn starch adhesive demonstrated significant erosion compared to the remaining adhesives. Without being bound by theory, it is understood that the pre-gelatinization of the starch significantly improves the wet integrity performance of the adhesive.

In use this results in the ostomy skin barrier device (1) comprising the medical adhesive (6) maintaining a seal around the stoma such that the patient's skin is not damaged by effluent.

FIG. 5 shows the change in the diameter of the aperture (8) of the ostomy device (1) (inner diameter change) when the medical adhesive (6) comprises inventive adhesive A, inventive adhesive B or control gelatin adhesive X, after exposure to a mixture of 1 g/L trypsin and 1 g/L amylase enzymes at a total enzyme concentration of 2 g/L in saline. Trypsin breaks down proteins and amylase breaks down starch. Control gelatin adhesive X significantly erodes when exposed to the enzymes whereas the inventive adhesive A and inventive adhesive B do not. This shows that inventive adhesive A and inventive adhesive B have greater enzyme resistance which in turn leads to a longer lasting medical adhesive wherein the adhesive maintains a seal around the stoma such that the patient's skin is not damaged by effluent.

FIG. 6 shows the average peel force of inventive adhesive A, inventive adhesive B and control gelatin adhesive X. The peel force was measured according to the following method. Medical adhesive samples comprising a polyethylene backing, the medical adhesive layer comprising inventive adhesive A, inventive adhesive B or control gelatin adhesive X, and a siliconized release paper were prepared. TESA 4590 (monofilament tape, width 25 mm) was placed on the polyethylene side of the sample using gentle pressure to adhere it. The medical adhesive sample was applied to a substrate using a ChemInstruments Rolldown machine and rolled one time at 60 cm/min. The substrate was either stainless steel or Teflon. The medical adhesive sample and substrate were placed onto a sled and the substrate was clamped. The force required to peel the medical adhesive sample from the substrate was measured. The peel rate was either 200 mm/minute or 600 mm/minute. Inventive adhesive A, inventive adhesive B and control gelatin adhesive X were tested according to the same conditions and the comparable data is presented in FIG. 6. The data shows that the peel force for inventive adhesive A, inventive adhesive B and control gelatin adhesive X are comparable; therefore, the peel performance of the medical adhesive is not derated by replacing a gelatin adhesive with a medical adhesive comprising pre-gelatinized acetylated distarch adipate or pre-gelatinized hydroxypropyl distarch phosphate.

FIG. 7 shows the maximum tack force of inventive adhesive A, inventive adhesive B, the control gelatin adhesive X. The tack force was measured using a tack testing probe with a 10N load cell. The test speed was 600 mm/minute with a hold time of 1 second. The inventive adhesives and the control adhesive were tested according to the same conditions and the comparable data is presented in FIG. 7. The data shows that the tack force for the inventive adhesive A, inventive adhesive B and control gelatin adhesive X are comparable; therefore, the tack performance of the medical adhesive is not derated by replacing a gelatin adhesive with a medical adhesive comprising pre-gelatinized acetylated distarch adipate or pre-gelatinized hydroxypropyl distarch phosphate.

In conclusion, the skin barrier device (1) of the invention comprising the inventive medical adhesive (6) is advantageous because it allows the skin barrier device (1), and therefore the ostomy bag (12), to be securely attached to the patient so that the patient can do everyday activities without discomfort. The inventive medical adhesive (6) is advantageous because it is gelatin free which allows it to be used on damaged or cracked skin with a reduced risk of infection whilst maintaining high water integrity, comparable water absorbance and comparable peel and tack performance.

In use this provides a gelatin free skin barrier device (1) which maintains adhesion to skin on exposure to water (such as exposure to sweat), maintains a seal around the patient's stoma and has greater enzyme resistance, all of which in turn result in a skin barrier device (1) with longer lasting adhesion, reduced leakage and reduced peristomal skin irritation, and therefore longer wear times.

A second embodiment of an ostomy skin barrier device (101) of the first aspect of the invention is illustrated in FIG. 8 and FIG. 9. The skin barrier device (101) comprises; a front face (102) and an opposing back face (104) wherein at least a portion or substantially all of the front face (102) comprises a medical adhesive (106), illustrated by a dotted line, wherein the medical adhesive (106) comprises a pre-gelatinized modified starch. The ostomy skin barrier device (101) comprises an aperture (108) suitable for at least partially receiving a stoma.

The medical adhesive (106) is according to the medical adhesive (6) of the first embodiment of the ostomy skin barrier device (1).

In use the front face (102) of the ostomy skin barrier device (101) is in contact with and adhered to the patient's peristomal skin by way of the inventive medical adhesive (106) and the patient's stoma may be at least partially inserted into the aperture (108). The ostomy skin barrier device (101) may then be in contact with a further skin barrier device, adhesive wafer or an ostomy pouch. The ostomy skin barrier device (101) is advantageous because it protects the patient's peristomal skin from abrasion or irritation. The ostomy skin barrier device (101) is particularly advantageous should the peri-stomal skin be uneven because the good wet integrity of the adhesive (106) ensures that once the skin barrier device is applied to the skin, a secure seal is formed, and the structural integrity of the adhesive is maintained on exposure to water and/or enzymes thereby reducing the occurrence of leakage or skin irritation.

A third embodiment of an ostomy skin barrier device of the first aspect of the invention is provided wherein the ostomy skin barrier device comprises; a front face and an opposing back face wherein at least a portion or substantially all of the front face comprises a medical adhesive, wherein the medical adhesive comprises a pre-gelatinized starch. The ostomy skin barrier device comprises an aperture suitable for at least partially receiving a stoma. The back face comprises a skin barrier device connector surrounding the aperture wherein the skin barrier device connector is suitable for connecting to the corresponding ostomy bag connector located on an ostomy bag.

The third embodiment of the ostomy skin barrier device is essentially the same as the first embodiment except for the medical adhesive comprises a pre-gelatinized starch instead of a pre-gelatinized modified starch. The pre-gelatinized starch is pre-gelatinized potato starch. The medical adhesive comprises: 20 wt. % pre-gelatinized potato starch; 20 wt. % pectin; 40 wt. % polyisobutylene; and 20 wt. % carboxymethyl cellulose.

A fourth embodiment of an ostomy skin barrier device of the first aspect of the invention is provided wherein the skin barrier device comprises; a front face and an opposing back face wherein at least a portion or substantially all of the front face comprises a medical adhesive, wherein the medical adhesive comprises a pre-gelatinized starch. The ostomy skin barrier device comprises an aperture suitable for at least partially receiving a stoma.

The fourth embodiment of the ostomy skin barrier device is essentially the same as the second embodiment except for the medical adhesive comprises a pre-gelatinized starch instead of a pre-gelatinized modified starch. The pre-gelatinized starch is pre-gelatinized potato starch. The medical adhesive comprises: 20 wt. % pre-gelatinized potato starch; 20 wt. % pectin; 40 wt. % polyisobutylene; and 20 wt. % carboxymethyl cellulose.

The above embodiments are described by way of example only. Many variations are possible without departing from the scope of the invention as defined in the appended claims.

1. An ostomy skin barrier device comprising;
a front face and a back face wherein at least a portion of the front face comprises a medical adhesive wherein the medical adhesive comprises a pre-gelatinized starch or a pre-gelatinized modified starch; and
an aperture in the skin barrier device for at least partially receiving a stoma, in use.

2. An ostomy skin barrier device according to claim 1 wherein the pre-gelatinized starch or the pre-gelatinized modified starch comprises no more than 5 wt. % amylose.

3. An ostomy skin barrier device according to claim 1 or claim 2 wherein the pre-gelatinized starch or the pre-gelatinized modified starch comprises at least 95 wt. % amylopectin.

4. An ostomy skin barrier device according to any preceding claim wherein the pre-gelatinized starch or the pre-gelatinized modified starch is not crosslinked.

5. An ostomy skin barrier device according to any of claims 1 to 3 wherein the pre-gelatinized starch or the pre-gelatinized modified starch is crosslinked.

6. An ostomy skin barrier device according to any preceding claim wherein the amount of the pre-gelatinized starch or pre-gelatinized modified starch is between 10 and 40 wt. % of the total weight of the adhesive.

7. An ostomy skin barrier device according to any preceding claim wherein the pre-gelatinized starch or pre-gelatinized modified starch is a pre-gelatinized distarch.

8. An ostomy skin barrier device according to any preceding claim wherein the pre-gelatinized starch or pre-gelatinized modified starch is a pre-gelatinized distarch adipate or a pre-gelatinized distarch phosphate.

9. An ostomy skin barrier device according to any preceding claim wherein the pre-gelatinized starch or pre-gelatinized modified starch is a pre-gelatinized acetylated distarch adipate or a pre-gelatinized hydroxypropyl distarch phosphate.

10. An ostomy skin barrier device according to any preceding claim wherein the medical adhesive comprises pectin.

11. An ostomy skin barrier device according to claim 10 wherein the amount of pectin is between 10 and 30 wt. % of the total weight of the adhesive.

12. An ostomy skin barrier device according to any preceding claim wherein the adhesive comprises polyisobutylene.

13. An ostomy skin barrier device according to claim 12 wherein the amount of polyisobutylene is between 5 and 50 wt. % of the total weight of the adhesive.

14. An ostomy skin barrier device according to any preceding claim wherein the adhesive composition comprises cellulose or a cellulose derivative.

15. An ostomy skin barrier device according to claim 14 wherein the cellulose derivative is a carboxymethyl cellulose.

16. An ostomy skin barrier device according claim 14 or claim 15 wherein the amount of the cellulose derivative is between 10 and 30 wt. % of the total weight of the adhesive.

17. An ostomy skin barrier device according to any preceding claim wherein the medical adhesive comprises;
between 10 and 40 wt. % pre-gelatinized starch or pre-gelatinized modified starch;
between 10 and 30 wt. % pectin;
between 5 and 50 wt. % polyisobutylene; and
between 10 and 30 wt. %. cellulose or a cellulose derivative.

18. An ostomy skin barrier device according to any preceding claim wherein the thickness of the adhesive layer is between 0.05 mm and 2 mm.

19. A medical adhesive comprising at least one starch or modified starch wherein the starch or modified starch comprises no more than 5 wt. % amylose.

20. A medical adhesive comprising at least one starch or modified starch wherein the starch or modified starch comprises at least 95 wt. % amylopectin.
